(19) United States
(12) Patent Application Publication
(10) Pub. No.: US 2017/0340893 A1
(43) Pub. Date: Nov. 30, 2017

(54) QUANTUM WAVE THERAPY APPARATUS

(71) Applicant: NEW ENERGY INDUSTRY CO., LTD., Osaka (JP)

(72) Inventor: Kiyonori SHINNOU, Osaka (JP)

(21) Appl. No.: 15/601,090

(22) Filed: May 22, 2017

(30) Foreign Application Priority Data
May 26, 2016 (JP) 2016-104906

(51) Int. Cl. A61N 1/40; A61N 2/00; A61N 5/00; A61N 5/06

(52) U.S. Cl. CPC A61N 1/40; A61N 5/00; A61N 2/00; A61N 2005/0664

(57) ABSTRACT

A quantum wave therapy apparatus includes a quantum wave generator and a quantum wave irradiator. The quantum wave irradiator includes an oscillation plate, and a resin panel that is formed by kneading semiconductor powder, which reflects quantum waves, into resin. The quantum wave generator includes an AC adaptor, and a coil that is connected in series to the negative electrode side of an output smoothing capacitor in the AC adaptor, and that constitutes a serial resonance circuit. An electromotive voltage induced in the serial resonance circuit by extraneous electromagnetic waves is superimposed on a negative potential output from the AC adaptor, and a resulting voltage is applied to the oscillation plate, whereby quantum waves are generated. The quantum waves are applied to a diseased portion. The quantum waves reflected from surroundings are reflected again by the semiconductor powder in the resin panel and are applied to the diseased portion repeatedly.

### BACKGROUND OF THE INVENTION

### 1. FIELD OF THE INVENTION

The present invention relates to a quantum wave therapy apparatus of applying quantum waves to a portion to be irradiated, such as a diseased portion of a human or an animal, thereby activating the irradiated portion for therapy.

### 2. DESCRIPTION OF THE RELATED ART

The above-mentioned type of quantum wave therapy apparatus is proposed, for example, by the inventor of this application as disclosed in Japanese Patent No. 5684434. According to the disclosed related art, an LC-parallel resonance circuit, a diode, and a safety resistance are disposed in series in one line of a commercial AC system. Such an arrangement generates a voltage resulting from superimposition of an electromotive voltage, which is induced in the resonance circuit by extraneous electromagnetic waves, on a commercial AC current. Of the generated voltage, a negative component is taken out through a diode and is applied to an oscillation plate that is held in contact with diseased tissues of a human body. Hence outer-electron orbits of material atoms constituting the oscillation plate are varied, thus causing vibrations of protons in the material atoms and hence generating quantum waves. A quantum is represented by a photon, an electron, a quark, a neutrino, etc.

By utilizing the quantum waves, a therapy apparatus is realized which is simpler and more inexpensive than therapy apparatuses that have been widely used so far by utilizing microwaves, electromagnetic waves, electric pulses and so on. The therapy apparatuses having been used so far have a comparatively high therapy effect for diseases close to skins, such as shoulder stiffness, bruise, and pains, but they hardly exhibit a therapy effect for diseases occurred in deep parts of a human body, such as cuts, rheumatism, nerve pain, diabetes, and enlargement of prostate. In contrast, the therapy apparatus disclosed in Japanese Patent No. 5684434 is more excellent because it is effective to the diseases occurred in the deep parts as well.

### SUMMARY OF THE INVENTION

Through efforts of studying many and various cases after fabricating the therapy apparatus disclosed in Japanese Patent No. 5684434, the inventor of this application has achieved an invention that can exhibit a higher therapy effect, can generate quantum waves with higher intensity, and can efficiently utilize the generated quantum waves.

An object of the present invention is to provide a quantum wave therapy apparatus having a higher therapy effect.

The present invention provides a quantum wave therapy apparatus including a quantum wave generator, and a quantum wave irradiator that is placed near a portion to be irradiated, and that applies quantum waves generated by the quantum wave generator to the irradiated portion, wherein the quantum wave generator includes an AC adaptor that rectifies and smooths a commercial AC current, and a coil that is connected in series to a negative electrode side of an output smoothing capacitor in the AC adaptor, and that constitutes a serial resonance circuit, and wherein the quantum wave irradiator includes an oscillation plate connected to the coil, and a resin panel that is formed by kneading semiconductor powder, which reflects the quantum waves, into resin, and that is attached to the oscillation plate to be interposed between the oscillation plate and the irradiated portion.

According to the features described above, in the quantum wave therapy apparatus of applying the quantum waves generated by the quantum wave generator to the irradiated portion, such as a diseased portion of a human or an animal, from the quantum wave irradiator, which is placed near the irradiated portion, thereby activating the irradiated portion for therapy, the generation and the irradiation of the quantum waves are performed as follows.

First, the quantum wave generator includes the AC adaptor that rectifies and smooths the commercial AC current, and the coil that is connected in series to the negative electrode side of the output smoothing capacitor in the AC adaptor, and that constitutes the serial resonance circuit. Therefore, an electromotive voltage induced in the serial resonance circuit by extraneous electromagnetic waves is superimposed on a potential at a negative electrode, and a resulting variation of the negative potential is applied to the oscillation plate. Hence outer-electron orbits of material atoms constituting the oscillation plate are varied, whereby the quantum waves can be generated efficiently due to vibrations of protons in the material atoms.

Furthermore, the quantum waves emitted from the oscillation plate are applied to the irradiated portion. On that occasion, the quantum waves having been reflected from the irradiated portion without permeating into the diseased portion, etc. are reflected again by the semiconductor powder in the resin panel and are applied to the irradiated portion repeatedly.

As a result, the quantum waves having been generated efficiently can be applied to the irradiated portion with no leakage, and a therapy effect can be enhanced.

The present invention further provides a quantum wave therapy apparatus including a quantum wave generator, and a quantum wave irradiator that is placed near a portion to be irradiated, and that applies quantum waves generated by the quantum wave generator to the irradiated portion, wherein the quantum wave generator includes an LC parallel resonance circuit disposed in one line of a single-phase two-wire commercial AC system, diodes disposed in series in both lines of the single-phase two-wire commercial AC system, respectively, with cathode sides of the diodes positioned on the side connected to a power supply of the commercial AC system, and a resistance to which anode sides of the diodes are connected in parallel, and wherein the quantum wave irradiator includes an oscillation plate connected to the resistance, and a resin panel that is formed by kneading semiconductor powder, which reflects the quantum waves, into resin, and that is attached to the oscillation plate to be interposed between the oscillation plate and the irradiated portion.

According to the features described above, in the quantum wave therapy apparatus of applying the quantum waves generated by the quantum wave generator to the irradiated portion, such as a diseased portion of a human or an animal, from the quantum wave irradiator, which is placed near the irradiated portion, thereby activating the irradiated portion for therapy, the generation and the irradiation of the quantum waves are performed as follows.

First, the quantum wave generator includes the LC parallel resonance circuit disposed in one line of the single-phase two-wire commercial AC system, the diodes disposed in series in both the lines of the single-phase two-wire commercial AC system, respectively, with the cathode sides of the diodes positioned on the side connected to the power supply of the commercial AC system, and the resistance to which the anode sides of the diodes are connected in parallel. Therefore, an electromotive voltage induced in the parallel resonance circuit by extraneous electromagnetic waves is superimposed on a commercial AC current, and a resulting variation of a negative potential is taken out through the diode and is applied to the oscillation plate through a safety resistance. Hence outer-electron orbits of material atoms constituting the oscillation plate are varied, whereby the quantum waves can be generated efficiently due to vibrations of protons in the material atoms.

Furthermore, the quantum waves emitted from the oscillation plate are applied to the irradiated portion. On that occasion, the quantum waves having been reflected from the irradiated portion without permeating into the diseased portion are reflected again by the semiconductor powder in the resin panel and are applied to the irradiated portion repeatedly.

As a result, the quantum waves having been generated efficiently can be applied to the irradiated portion with no leakage, and a therapy effect can be enhanced.

In the quantum wave therapy apparatus of the present invention, a resonance frequency of the resonance circuit is 2 to 2000 Hz and preferably 2 to 100 Hz.

According to the feature described above, an effect of promoting blood circulation in a human body can be obtained, which makes it possible to relieve shoulder stiffness and swelling, or to cure wounds in a shorter time.

In the quantum wave therapy apparatus of the present invention, a content of the semiconductor powder is 2 to 20% by weight and in particular preferably 5% by weight.

The above-described feature is based on results of experiments conducted by the inventor of this application, the results showing that, with respect to concentration of the semiconductor powder, reflectance of the quantum waves starts to saturate at about 2% by weight. Thus, a minimum content of the semiconductor powder is set to 2% by weight, which also contributes to increasing the cost performance. Taking into account variations of concentration and so on, an optimum value is preferably set to 5% by weight. On the other hand, because the reflectance of the quantum waves are almost completely saturated at 20% by weight, a maximum value is preferably set to 20% by weight from the viewpoint of not only reducing the material cost, but also suppressing reduction in strength of the resin.

In the quantum wave therapy apparatus of the present invention, when the content of the semiconductor powder is 5% by weight, the resin panel is formed as a plate having a thickness of 2 mm or more.

In relation to the above-described feature, increasing the thickness of the resin panel enhances an effect of reflecting the quantum waves, but increases the cost.

By setting the thickness of the resin panel to 2 mm or more, the reflectance is given at an intended level that is obtained when the content of the semiconductor powder is 5% by weight. Moreover, by setting the thickness of the resin panel to 2 mm, the cost performance can be increased.

In the quantum wave therapy apparatus of the present invention, the semiconductor powder is made of a metal oxide and/or a silicate compound.

The above-described feature is based on results of experiments conducted by the inventor of this application, the results showing that when the semiconductor powder is made of a metal oxide and/or a silicate compound, the effect of reflecting the quantum waves is enhanced. The metal oxide preferably contains at least one of zinc oxide, titanium oxide, zirconium oxide, and aluminum oxide. In addition, the metal oxide and the silicate compound are preferably used in combination of plural types.

In the quantum wave therapy apparatus of the present invention, metal powder for giving electrical conductivity is further kneaded into the resin.

According to the feature described above, since the metal powder for giving electrical conductivity to the semiconductor powder made of the metal oxide and/or the silicate compound is further kneaded into the resin, the effect of reflecting the quantum waves can be further enhanced.

In the quantum wave therapy apparatus of the present invention, the semiconductor powder and the metal powder are powders of 10 to 100 μm and in particular preferably 30 to 50 μm.

According to the feature described above, the effect of reflecting the quantum waves can be further enhanced.

### BRIEF DESCRIPTION OF THE DRAWINGS

FIG. 1 schematically illustrates a configuration and usage of a quantum wave therapy apparatus according to one embodiment of the present invention;

FIG. 2 is a sectional view of a quantum wave irradiator used in the quantum wave therapy apparatus; and

FIG. 3 schematically illustrates a configuration and usage of a quantum wave therapy apparatus according to another embodiment of the present invention.

### DESCRIPTION OF THE PREFERRED EMBODIMENTS

### First Embodiment

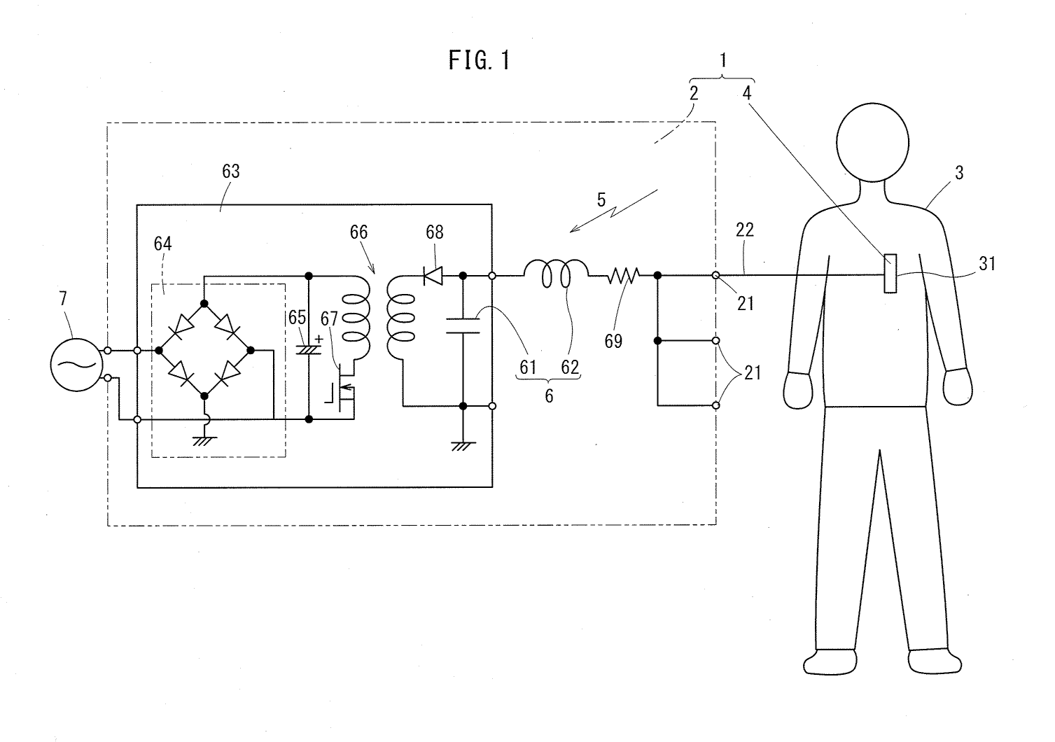

FIG. 1 schematically illustrates a configuration and usage of a quantum wave therapy apparatus 1 according to one embodiment of the present invention. The quantum wave therapy apparatus 1 is intended to apply quantum waves, which are generated by a quantum wave generator 2, through a quantum wave irradiator 4 held in contact with a portion to be irradiated, such as a diseased portion 31 of a human body 3, thereby activating the diseased portion 31 for therapy. The quantum wave irradiator 4 may be held in direct contact with the diseased portion 31 where therapy is to be given, or may be just placed near the diseased portion 31 on condition that a spacing therebetween is several centimeters.

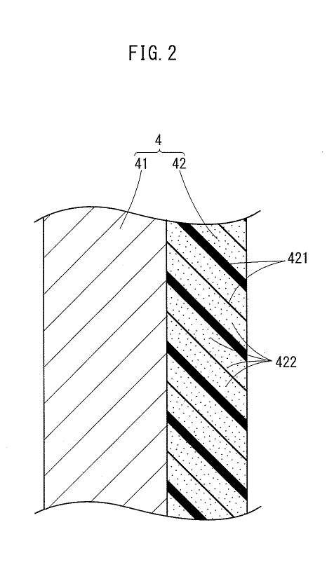

A generation mechanism of quantum waves is as follows. An electromotive voltage induced in a serial resonance circuit 6 by extraneous electromagnetic waves 5 is superimposed on a negative potential, and a variation of the negative potential of a resulting voltage is applied to an oscillation plate 41 (see FIG. 2) of the quantum wave irradiator 4. Hence outer-electron orbits of material atoms constituting the oscillation plate 41 are varied, whereby quantum waves are emitted due to vibrations of protons (plus ions) in the material atoms. This accelerates vibrations of molecules and atoms in tissues of the diseased portion 31 that is positioned in contact with or near the oscillation plate 41.

Moreover, when an electric field near the oscillation plate 41 varies with the above-described potential variation, groups of molecules and atoms constituting biological tissues repeat vibrations. In the case of water or a liquid, activity of the water or the liquid is changed because of, for example, reduction in size of clusters. More specifically, when a frequency of the electromotive force induced in the serial resonance circuit 6 resonates with the vibrations of molecules and atoms in biological tissues, activation of the biological tissues is promoted. As a result, promotion of blood circulation and metabolism are enhanced. Furthermore, because electrons are added with the variation of the negative electric field, a reducing character of water in the biological tissues is increased, and oxidization of the biological tissues is suppressed. Hence an effect of preventing fester, for example, is obtained.

Here, a resonance frequency of the serial resonance circuit 6 is 2 to 2000 Hz and preferably 2 to 100 Hz. Thus, the resonance frequency is very low, and the capacitance of a capacitor 61 and the inductance of a coil 62, which constitute the serial resonance circuit 6, are comparatively large. In this embodiment, therefore, an output smoothing capacitor in an AC adaptor 63 is used as the capacitor 61 constituting the serial resonance circuit 6.

The AC adaptor 63 includes a rectifier circuit 64, a smoothing capacitor 65, a high-frequency transformer 66, a switching element 67, a diode 68, and the above-mentioned capacitor 61.

The rectifier circuit 64 performs full-wave rectification of an AC current supplied from a commercial power supply 7. A pulsating current resulting from the rectification is smoothed by the smoothing capacitor 65. A primary winding of the high-frequency transformer 66 and the switching element 67 are connected in series between terminals of the smoothing capacitor 65. Accordingly, a voltage is induced in a secondary winding of the high-frequency transformer 66 upon switching of the switching element 67 with the smoothing capacitor 65 serving as a power source, and only a negative component of the induced voltage is taken out from an anode of the diode 68 of which cathode is connected to the secondary winding. The taken-out negative component is smoothed by and output from the capacitor 61. An output voltage of the AC adaptor 63 is, for example, 12 to 48 V.

A coil 62 is connected in series to a negative polarity side of the capacitor 61 in the AC adaptor 63, thus constituting the serial resonance circuit 6. The electromotive voltage induced in the serial resonance circuit 6 by the extraneous electromagnetic waves 5 is superimposed on a negative potential output from the AC adaptor 63 (i.e., on the negative component taken out from the diode 68), and a resulting voltage is output from a terminal 21 through a safety resistance 69. The oscillation plate 41 is connected to the terminal 21 through a cable 22. In the quantum wave generator 2, multiple terminals 21 are disposed in parallel.

Referring to FIG. 2, the quantum wave irradiator 4 includes the oscillation plate 41, and a resin panel 42 that is attached to a surface of the oscillation plate 41 to be interposed between the diseased portion 31 and the oscillation plate 41. The oscillation plate 41 is a metal plate made of aluminum, stainless steel, titanium, or iron, for example. It is noteworthy that the resin panel 42 is constituted by kneading semiconductor powder 422, which reflects the quantum waves, into a resin 421.

According to the quantum wave therapy apparatus 1 constituted as described above, the voltage resulting from superimposition of the electromotive voltage, which is induced in the serial resonance circuit 6 by the extraneous electromagnetic waves 5, on the negative potential taken out from the AC adaptor 63 is applied to the oscillation plate 41, and hence the quantum waves can be generated efficiently in the oscillation plate 41. Furthermore, the quantum waves emitted from the oscillation plate 41 are applied to the diseased portion 31. On that occasion, the quantum waves having been reflected without permeating into the diseased portion 31 are reflected again by the semiconductor powder 422 in the resin panel 42 and are applied to the diseased portion 31 repeatedly. Thus, the quantum waves having been generated efficiently can be applied to the diseased portion 31 with no leakage, and a therapy effect can be enhanced.

Since the resonance frequency of the serial resonance circuit 6 is 2 to 2000 Hz and preferably 2 to 100 Hz, an effect of promoting blood circulation in a human body can be obtained, which makes it possible to relieve shoulder stiffness and swelling, or to cure wounds in a shorter time. For providing the resonance frequency of the above range, the coil 62 has an inductance of 1000 to 1500 mH, for example.

Regarding the quantum wave irradiator 4 constituted as described above, the results of experiments conducted by the inventor of this application show that, with respect to concentration of the semiconductor powder 422, reflectance of the quantum waves starts to saturate at about 2% by weight. Therefore, a minimum content of the semiconductor powder 422 is preferably set to 2% by weight. This also contributes to increasing the cost performance. Taking into account variations of concentration and so on, it is particularly preferable that an optimum value is set to 5% by weight. On the other hand, because the reflectance of the quantum waves are almost completely saturated at 20% by weight, a maximum value is preferably set to 20% by weight from the viewpoint of not only reducing the material cost, but also suppressing reduction in strength of the resin 421.

The resin panel 42 is formed by kneading the semiconductor powder 422 into the resin 421 such as ABS, and by molding the resin. Increasing a thickness of the resin panel 42 enhances the effect of reflecting the quantum waves, but increases the cost. By setting the thickness of the resin panel 42 to 2 mm or more, the reflectance is given at an intended level that is obtained when the semiconductor powder 422 is contained at the above-mentioned optimum value of 5% by weight. By setting the thickness of the resin panel 42 to 2 mm that is the least necessary thickness, the cost performance can be increased.

Furthermore, the results of experiments conducted by the inventor of this application show that, as materials of the semiconductor powder 422, a metal oxide and a silicate compound are preferable because the effect of reflecting the quantum waves is relatively large. The metal oxide preferably contains at least one of zinc oxide, titanium oxide, zirconium oxide, and aluminum oxide. An example of the silicate compound is magnesium silicate. The metal oxide and the silicate compound are preferably used in combination of plural types. Moreover, the metal oxide and the silicate compound may be used in not only in a state of refined minerals, but also in a state obtained by pulverizing ores containing them in the form of natural minerals.

In addition, metal powder for giving electrical conductivity is preferably kneaded into the resin 421. The metal powder is powder of tin or zinc, for example. With the metal powder kneaded into the resin 421, the effect of reflecting the quantum waves by the resin panel 42 can be enhanced. The effect of reflecting the quantum waves by the resin panel 42 can be further enhanced by preparing the semiconductor powder 422 and the metal powder as powders of not less than 10 μm and not more than 100 μm and in particular preferably not less than 30 μm and not more than 50 μm (e.g., 325 mesh).

### Second Embodiment

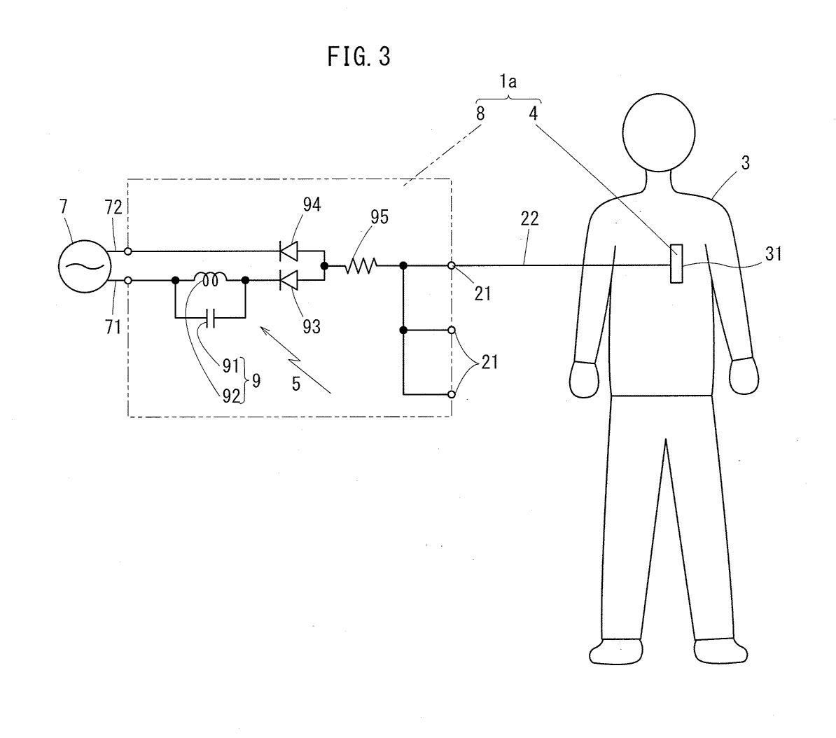

FIG. 3 schematically illustrates a configuration and usage of a quantum wave therapy apparatus la according to another embodiment of the present invention. The quantum wave therapy apparatus la is different from the above-described quantum wave therapy apparatus 1 only in a quantum wave generator 8, and a quantum wave irradiator 4 in the quantum wave therapy apparatus 1a is the same as that in the quantum wave therapy apparatus 1. Corresponding components of the quantum wave therapy apparatus la to those in the quantum wave therapy apparatus 1 are denoted by same reference signs, and description of those components is omitted.

The quantum wave generator 8 includes an LC parallel resonance circuit 9 that is disposed in one AC line 71 extending from a single-phase two-wire commercial power supply 7, diodes 93 and 94 that are disposed respectively in AC lines 71 and 72 of a single-phase two-wire system in series with their cathode sides positioned closer to the commercial power supply 7, and a safety resistance 95 to which the anode sides of the diodes 93 and 94 are connected in parallel. The LC parallel resonance circuit 9 includes a capacitor 91 and a coil 92, and has a resonance frequency of 2 to 2000 Hz and preferably 2 to 100 Hz as in the above-described embodiment.

With such a circuit configuration, an electromotive voltage induced in the LC parallel resonance circuit 9 by the extraneous electromagnetic waves 5 is superimposed on a variation of a negative potential (effective value of 100 V) that is obtained with the diodes 93 and 94, and a resulting variation of the negative potential is applied to the oscillation plate 41 from the safety resistance 95 through the terminal 21 and the cable 22. Thus, the quantum waves are generated as in the above-described embodiment. In FIG. 1 of the above-cited PTL 1, only one (corresponding to 71 in this embodiment) of two lines extending from a commercial power supply is used. In contrast, in this embodiment, the diode 94 is additionally disposed in the other line 72 and the anode side of the diode 94 is connected to the safety resistance 95 in common with the diode 93 in order that the intended effect can be obtained regardless of which one of the lines 71 and 72 is grounded in a pole-mounted transformer.

With the configuration described above, it is also possible to apply the quantum waves, which have been generated efficiently, to the diseased portion 31 without leakage, and to enhance the therapy effect. Furthermore, when the resonance frequency of the LC parallel resonance circuit 9 is high, a varying electric field at a high frequency and a varying electric field at a low (commercial) frequency act on the diseased portion 31 in a synergetic manner, and a higher synergetic effect can be developed in the therapy of the diseased portion 31.

What is claimed is:

1. A quantum wave therapy apparatus comprising a quantum wave generator, and a quantum wave irradiator that is placed near a portion to be irradiated, and that applies quantum waves generated by the quantum wave generator to the irradiated portion,
wherein the quantum wave generator comprises:
an AC adaptor that rectifies and smooths a commercial AC current; and
a coil that is connected in series to a negative electrode side of an output smoothing capacitor in the AC adaptor, and that constitutes a serial resonance circuit, and
wherein the quantum wave irradiator comprises:
an oscillation plate connected to the coil; and
a resin panel that is formed by kneading semiconductor powder, which reflects the quantum waves, into resin, and that is attached to the oscillation plate to be interposed between the oscillation plate and the irradiated portion.

2. A quantum wave therapy apparatus comprising a quantum wave generator, and a quantum wave irradiator that is placed near a portion to be irradiated, and that applies quantum waves generated by the quantum wave generator to the irradiated portion,
wherein the quantum wave generator comprises:
an LC parallel resonance circuit disposed in one line of a single-phase two-wire commercial AC system;
diodes disposed in series in both lines of the single-phase two-wire commercial AC system, respectively, with cathode sides of the diodes positioned on side connected to a power supply of the commercial AC system; and
a resistance to which anode sides of the diodes are connected in parallel, and
wherein the quantum wave irradiator comprises:
an oscillation plate connected to the resistance; and
a resin panel that is formed by kneading semiconductor powder, which reflects the quantum waves, into resin, and that is attached to the oscillation plate to be interposed between the oscillation plate and the irradiated portion.

3. The quantum wave therapy apparatus to claim 1, wherein a resonance frequency of the resonance circuit is 2 to 2000 Hz.

4. The quantum wave therapy apparatus to claim 2, wherein a resonance frequency of the resonance circuit is 2 to 2000 Hz.

5. The quantum wave therapy apparatus to claim 1, wherein a content of the semiconductor powder is 2 to 20% by weight.

6. The quantum wave therapy apparatus to claim 2, wherein a content of the semiconductor powder is 2 to 20% by weight.

7. The quantum wave therapy apparatus to claim 5, wherein, when the content of the semiconductor powder is 5% by weight, and the resin panel is formed as a plate having a thickness of 2 mm or more.

8. The quantum wave therapy apparatus to claim 6, wherein, when the content of the semiconductor powder is 5% by weight, and the resin panel is formed as a plate having a thickness of 2 mm or more.

9. The quantum wave therapy apparatus to claim 1, wherein the semiconductor powder is made of a metal oxide and/or a silicate compound.

10. The quantum wave therapy apparatus to claim 2, wherein the semiconductor powder is made of a metal oxide and/or a silicate compound.

11. The quantum wave therapy apparatus to claim 9, wherein the metal oxide contains at least one of zinc oxide, titanium oxide, zirconium oxide, and aluminum oxide.

12. The quantum wave therapy apparatus to claim 10, wherein the metal oxide contains at least one of zinc oxide, titanium oxide, zirconium oxide, and aluminum oxide.

13. The quantum wave therapy apparatus to claim 1, wherein metal powder for giving electrical conductivity is further kneaded into the resin.

14. The quantum wave therapy apparatus to claim 2, wherein metal powder for giving electrical conductivity is further kneaded into the resin.

15. The quantum wave therapy apparatus to claim 13, wherein the semiconductor powder and the metal powder are powders of 10 to 100 μm.

16. The quantum wave therapy apparatus to claim 14, wherein the semiconductor powder and the metal powder are powders of 10 to 100 μm.
